SEQUENCE LISTING (3 sequences)

SEQ ID NO: 1 - DNA, 75 bp, Artificial Sequence
  Synthetic construct
ctgttctgccccttcttttgatccggttgagctgctgagagtccaagagtgctcaaccggctgttctgccccttc

SEQ ID NO: 2 - DNA, 10 bp, Artificial Sequence
  Synthetic construct
ccggttgagc

SEQ ID NO: 3 - DNA, 46 bp, Artificial Sequence
  Synthetic construct
ggtcttacacaagaggactcttggactctcagcaatgtcaacgacc

(19) United States
(12) Patent Application Publication
(10) Pub. No.: US 2021/0040557 A1
(43) Pub. Date: Feb. 11, 2021

(54) TARGET NUCLEIC ACID-DETECTING METHOD USING HAIRPIN PROBE-ASSISTED ISOTHERMAL PROBE AMPLIFICATION

(71) Applicants: KOREA ADVANCED INSTITUTE OF SCIENCE AND TECHNOLOGY, Daejeon (KR); BIONANO HEALTH GUARD RESEARCH CENTER, Daejeon (KR)

(72) Inventors: Hyun Gyu PARK, Daejeon (KR); HyoYong KIM, Daejeon (KR); Jun Ki AHN, Daejeon (KR); Chang Yeol LEE, Daejeon (KR)

(21) Appl. No.: 16/966,778

(22) PCT Filed: Jan. 30, 2019
(86) PCT No.: PCT/KR2019/001255

§ 371 Date: Jul. 31, 2020

(30) Foreign Application Priority Data
Jan. 31, 2018 (KR) 10-2018-0012154

(51) Int. Cl. C12Q 1/6876

(52) U.S. Cl. CPC C12Q 1/6876

(57) ABSTRACT

The present invention relates to a target nucleic acid-detecting method using hairpin probe-assisted isothermal probe amplification (HIPAmp). According to the present invention, the method has the advantage of being able to conduct detection with respect to general-use target nucleic acids by solving the problem with the conventional isothermal nucleic acid amplification technique EXPAR with regard to limited uses of target nucleic acids.

### TECHNICAL FIELD

The present invention relates to a method for detecting a target nucleic acid using hairpin probe-assisted isothermal probe amplification (HIPAmp), and more specifically, a method for detecting a target nucleic acid using nucleic acid amplification including a hairpin ring having a base sequence specifically capable of binding to the target nucleic acid, hairpin stems having a base sequence capable of binding to a primer and being capable of binding complementarily to each other, cTP having a sequence complementary to a trigger probe (TP), and a cleavage enzyme recognition base sequence located between the stem and the cTP.

### BACKGROUND ART

Polymerase chain reaction (PCR) is the most widely used method for amplification and detection of target nucleic acids. However, precise temperature control is required in order to implement the PCR reaction, and there are disadvantages in that the volume of the PCR equipment is increased and costs are increased due to the installation of a temperature control device for this purpose. As alternatives to solve the disadvantages of the conventional PCR, various isothermal nucleic acid amplification methods such as strand displacement amplification (SDA), loop-mediated amplification (LAMP), nucleic-acid-sequence-based amplification (NASB), signal-mediated amplification of RNA technology (SMART), rolling circle amplification (RCA), helicase-dependent amplification (HDA), isothermal chain amplification (ICA), and recombinase polymerase amplification (RPA) have been active developed.

Among the isothermal nucleic acid amplification methods developed to date, EXPAR is receiving much attention as a representative isothermal nucleic acid amplification method having a fast detection time and high sensitivity, and the development of various biomaterial detection technologies based on EXPAR reactions is also being conducted. EXPAR is a method of detecting a target nucleic acid using a template specifically capable of binding to a target nucleic acid, based on a chain reaction of a DNA polymerase and a cleavage enzyme (Van Ness et al., Proc. Natl. Acad. Sci. U.S.A, 100, 4504, 2003).

More specifically, the template includes a cleavage enzyme recognition base sequence and the two same base sequence complementary to target nucleic acids linked to both ends of the cleavage enzyme recognition base sequence. When a target nucleic acid is present, a binding reaction between the template and the target nucleic acid occurs, and then, a synthesis reaction of new DNA proceeds from the target nucleic acid bound to the template by the activity of the DNA polymerase, and finally, a dsDNA product is produced from the template. In addition, the DNA strand is continuously produced by a chain reaction of a cleavage enzyme and a DNA polymerase, based on the cleavage enzyme recognition base sequence included in the obtained dsDNA product. The produced DNA strand may have the same nucleotide sequence as the target nucleic acid, and may bind to a template that does not participate in the reaction to thereby induce a reaction for obtaining a dsDNA product. As a result, an exponential amplification reaction for obtaining the dsDNA product is realized. However, in order to realize the EXPAR reaction described above, the synthesis of a template specific for the target nucleic acid is required, and moreover, the target nucleic acid is used directly as a substrate for the DNA synthesis reaction through the activity of the DNA polymerase, and thus EXPAR has a disadvantage in that it can be limitedly used only for the detection of short DNA strands (hereinafter referred to as DNA fragments).

In order to extend the range of utilization of EXPAR, a method for producing a DNA fragment that can be used as a substrate for the EXPAR reaction from a long target nucleic acid (hereinafter, referred to as a fingerprinting reaction) was devised. More specifically, the fingerprinting reaction is a method for producing a DNA fragment from a cleavage enzyme recognition base sequence present inside the target nucleic acid and a peripheral sequence containing the cleavage enzyme recognition base sequence based on a chain reaction of a cleavage enzyme and a DNA polymerase. The scope of utilization of the conventional EXPAR method has increased through the development of the present method. However, the fingerprinting reaction is a reaction that requires the cleavage enzyme recognition nucleotide sequence inside the target nucleic acid and thus is limitedly applied. Thus, the limitations on the use of EXPAR remain completely unsolved.

Accordingly, the present inventors endeavored to develop a method capable of overcoming the drawback of the limited range of utilization of conventional EXPAR by solving the problems associated with the prior art described above. As a result, the present inventors found that, when a nucleic acid is isothermally amplified, based on a probe having a hairpin structure capable of specifically binding to a certain base sequence inside the target nucleic acid, instead of the linear template used in the conventional EXPAR method, the target nucleic acid is not used as a direct substrate for a DNA polymerase reaction and amplification is thus possible even when there is no cleavage enzyme recognition sequence inside the target nucleic acid, and the target nucleic acid can be universally detected without limitation, and completed the present invention based on this finding.

### DISCLOSURE

### Technical Problem

It is one object of the present invention to provide a hairpin probe having a hairpin structure capable of specifically binding to a certain base sequence inside a target nucleic acid.

It is another object of the present invention to provide a method for detecting a target nucleic acid using the hairpin probe.

### Technical Solution

In accordance with one aspect of the present invention, the above and other objects can be accomplished by the provision of a hairpin probe for detecting a target nucleic acid including: (i) a hairpin ring having a base sequence specifically capable of binding to a target nucleic acid;

(ii) hairpin stems having a base sequence capable of binding to a primer and being capable of complementarily binding to each other, wherein each hairpin stem is linked to each end of the hairpin ring;

(iii) cTP linked to each end of hairpin stems of (b) and having a sequence complementary to a trigger probe (TP); and

(iv) a cleavage enzyme recognition base sequence located between the hairpin stem and cTP of the 5′ end of the hairpin probe.

In accordance with another aspect of the present invention, provided is a method for detecting a target nucleic acid including: (a) reacting a composition containing a target nucleic acid-containing sample, the hairpin probe, a primer capable of binding to the stem of the hairpin probe, a DNA polymerase, and dNTP to amplify dsDNA, and (d) analyzing the amplified dsDNA to detect a target nucleic acid.

In accordance with another aspect of the present invention, provided is a composition for detecting a target nucleic acid comprising a target nucleic acid-containing sample, the hairpin probe, a primer capable of binding to the stem of the hairpin probe, a DNA polymerase, and dNTP.

### DESCRIPTION OF DRAWINGS

FIG. 1 is a schematic diagram illustrating a reaction of a HIPAmp method according to the present invention, more particularly, (A) showing the reaction of producing IP 1 from a target nucleic acid, (B) showing the production and the detection reaction of a dsDNA product from the IP 1 produced in (A) above, and (C) showing the production and detection reaction of the dsDNA product induced by the binding reaction between the hairpin probe and the TP produced in (B).

FIG. 2 shows the experimental results found through real-time fluorescence signals and electrophoresis confirming the results of HIPAmp reaction of the present invention under various reaction conditions (1: reaction conditions excluding addition of hairpin probe and primer; 2: reaction conditions excluding addition of hairpin probe; 3: reaction conditions excluding addition of primer; 4: reaction conditions including addition of both hairpin probe and primer; and 5: reaction conditions excluding addition of target nucleic acid).

FIG. 3 shows the results of an experiment on the target nucleic acid detection sensitivity of the HIPAmp method according to the present invention, more particularly, (a) showing a real-time fluorescence signal generated from a reaction sample including a variety of target nucleic acid concentrations and (b) showing a linear relationship between a threshold time (hereinafter, referred to as t_{THR}) and a target nucleic acid concentration.

FIG. 4 shows the results of an experiment on the target nucleic acid detection selectivity of the HIPAmp method of the present invention based on the degree of a t_{THR} decrease value.

FIG. 5 shows the results of an experiment of real-time fluorescence signal measurement on samples containing target nucleic acids (2 nM) having various lengths (46, 208 and 895 bp) to determine the practicality of target nucleic acid detection of the HIPAmp method according to the present invention.

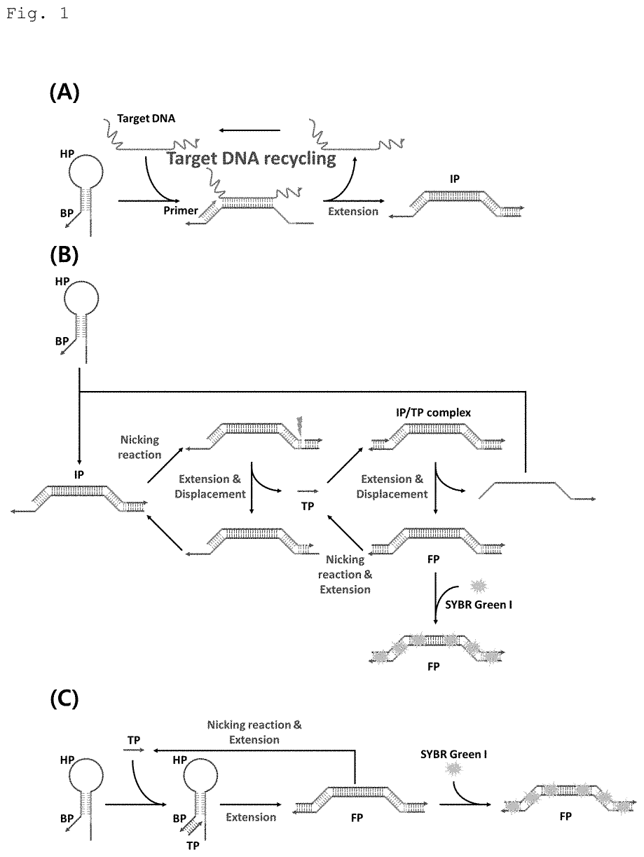

### BEST MODE

Unless defined otherwise, all technical and scientific terms used herein have the same meanings as appreciated by those skilled in the field to which the present invention pertains. In general, the nomenclature used herein is well-known in the art and is ordinarily used.

The method for detecting a target nucleic acid using hairpin probe-assisted isothermal probe amplification (hereinafter, referred to as HIPAmp) provided according to the present invention is a detection method capable of solving the disadvantages of the conventional EXPAR. More specifically, conventional EXPAR requires the synthesis of a template specific for the target nucleic acid and the direct use of the target nucleic acid as a substrate for the DNA synthesis reaction by DNA polymerase activity, thus being of limited usefulness for the detection of DNA fragments and ultimately having the disadvantage of being of limited usefulness of the technology. However, the HIPAmp according to the present invention solves the problem of limited usefulness of conventional EXPAR by introducing a hairpin probe capable of specifically binding to a certain base sequence inside the target nucleic acid, instead of the linear template used for conventional EXPAR. More specifically, the reaction system based on the hairpin probe is characterized in that the target nucleic acid is not used as a direct substrate for the enzymatic reaction, and when structural modification of the hairpin probe occurs due to binding of the hairpin probe to the target nucleic acid, the subsequent dsDNA product generation reaction system is operated. Therefore, the HIPAmp of the present invention is significant in that it is an isothermal nucleic acid amplification method that introduces a novel concept for solving the problem of limited usefulness of conventional EXPAR.

Accordingly, in one aspect, the present invention provides a hairpin probe for detecting a target nucleic acid comprising: (i) a hairpin ring having a base sequence specifically capable of binding to a target nucleic acid; (ii) hairpin stems having a base sequence capable of binding to a primer and being capable of complementarily binding to each other, wherein each hairpin stem is linked to each end of the hairpin ring; (iii) cTPs linked to each end of hairpin stems of (b) and having a sequence complementary to a trigger probe (TP); and (iv) a cleavage enzyme recognition base sequence located between the stem and cTP of the 5′ end of the hairpin probe.

In the HIPAmp method of the present invention, a hairpin probe capable of specifically binding to a certain base sequence inside the target nucleic acid was introduced, instead of the linear template used in the conventional EXPAR method. The hairpin probe includes a hairpin ring specifically capable of binding to a target nucleic acid, hairpin stems connected to both ends of the hairpin ring, being capable of binding to a primer and being capable of binding complementarily to each other; a base sequence (complementary TP, hereinafter referred to as “cTP”) connected to the end of the hairpin stem and capable of binding to a trigger probe (TP), and a cleavage enzyme recognition base sequence interposed between the hairpin stem and cTP of the 5′ end. When the target nucleic acid is present, bonds between the hairpin probe stems are broken due to the binding reaction between the ring portion of the hairpin probe and the target nucleic acid. This structural modification of the hairpin probe causes the primer included in the sample to bind to the stem portion of the hairpin probe having the modified structure.

Thereafter, the synthesis reaction of the new DNA starts from the bound primer due to the activity of the DNA polymerase, and the first reaction intermediate product IP 1 is produced. TP is produced from the produced IP 1 by a chain reaction of a cleavage enzyme and a DNA polymerase, and the produced TP may bind again to a single-stranded portion of IP 1. New DNA is synthesized from the TP bound to IP 1 by the activity of the DNA polymerase, resulting in a dsDNA product.

In another aspect, the present invention provides a method for detecting a target nucleic acid comprising: (a) reacting a composition comprising a target nucleic acid-containing sample, the hairpin probe, a primer capable of binding to the stem of the hairpin probe, a DNA polymerase and dNTP to amplify dsDNA; and (d) analyzing the amplified dsDNA to detect a target nucleic acid.

The method for detecting a target nucleic acid according to the present invention is conducted in accordance with the following procedure:

(a) binding a hairpin ring of a hairpin probe to a target nucleic acid, and binding a primer to a stem of the hairpin probe to form a complex of hairpin probe/target nucleic acid/primer;

(b) synthesizing new DNA from the primer by a DNA polymerase and separating the target nucleic acid from the complex of hairpin probe/target nucleic acid/primer to form a dsDNA intermediate product 1 (IP 1);

(c) binding the target nucleic acid separated in step (b) to a hairpin probe that does not participate in the reaction and repeating the steps (a) to (b) to amplify the production of the dsDNA, IP 1; and

(d) identifying the production of the IP 1 to detect the target nucleic acid.

In the present invention, the DNA polymerase may have strand displacement activity to displace DNA bound to a template.

In the present invention, the detection may be performed by detecting a fluorescence signal generated by binding of IP 1 to a fluorescent substance specifically binding to dsDNA.

In the dsDNA product production reaction, a second reaction intermediate product, IP 2 is produced, and the IP 2 binds to a hairpin probe that does not participate in the reaction to form IP 1.

The DNA polymerase used in the HIPAmp has strand displacement activity to displace DNA bound to a template (strand displacement activity) in the DNA synthesis reaction process. Accordingly, it is possible to configure a reaction system for recycling target nucleic acids and reaction intermediate products (IP 1 & IP 2) based on the activity of the DNA polymerase, and consequently, to increase the efficiency of the dsDNA product production reaction.

The method for detecting a target nucleic acid according to the present invention may be also conducted in accordance with the following procedure:

(a) binding a hairpin ring of a hairpin probe to a target nucleic acid, and binding a primer to a stem of the hairpin probe to form a complex of hairpin probe/target nucleic acid/primer;

(b) synthesizing new DNA from the primer by a DNA polymerase and separating the target nucleic acid from the complex of hairpin probe/target nucleic acid/primer to form a dsDNA intermediate product 1 (IP 1);

(c) binding the target nucleic acid separated in step (b) to a hairpin probe that does not participate in the reaction and repeating the steps (a) to (b) to amplify the production of IP 1;

(d) continuously forming a trigger probe (TP) from the IP 1 by a cleavage enzyme;

(e) binding the formed TP to a single-stranded portion of IP 1 and conducting a DNA synthesis reaction from the TP by a DNA polymerase to form a dsDNA product and, at the same time, to separate intermediate product (IP 2), a newly produced DNA strand of the dsDNA product, from the IP 1;

(f) continuously producing TP from the dsDNA product formed in step (e) by a cleavage enzyme and a DNA polymerase;

(g) binding the IP 2 formed in step (e) to a hairpin probe that does not participate in the reaction and repeating the steps (e) to (f) to amplify the production of a dsDNA product; and

(h) identifying the dsDNA to detect the target nucleic acid.

In the present invention, the DNA polymerase may have strand displacement activity to displace DNA bound to a template.

In the present invention, the detection may be performed by detecting a fluorescence signal generated by binding to a fluorescent substance specifically binding to dsDNA.

In addition, the TP produced in the reaction may directly bind to a hairpin probe that does not participate in the reaction, and when a DNA synthesis reaction proceeds from TP bound to the hairpin probe by a DNA polymerase, a dsDNA product may be produced.

In one embodiment of the present invention, the following probe of SEQ ID NO: 1 is used as an example of the hairpin probe, but the invention is not limited thereto.

Hairpin probe:
(SEQ ID NO: 1)
5′-CTG TTC TGC CCC TTC TTT TGA T [symbol] GGT TGA GCT
GCT GAG AGT CCA AGA GTG CTC AAC CGG CTG TTG TGC
CCC TTC-3′

In the base sequence of the hairpin probe, the base sequences indicated by underlining, italics, and boldface represent the base sequences of the cCP, the cleavage enzyme recognition site and the hairpin stem, respectively.

Accordingly, the detection method according to the present invention may be conducted in accordance with the following procedure:

(a) binding a hairpin ring of a hairpin probe to a target nucleic acid, and binding a primer to a stem of the hairpin probe to form a complex of hairpin probe/target nucleic acid/primer;

(b) synthesizing new DNA from the primer by a DNA polymerase and separating the target nucleic acid from the complex of hairpin probe/target nucleic acid/primer to form a dsDNA intermediate product 1 (IP 1);

(c) binding the target nucleic acid separated in step (b) to a hairpin probe that does not participate in the reaction and repeating the steps (a) to (b) to amplify the production of IP 1;

(d) continuously forming a trigger probe (TP) from the IP by a cleavage enzyme;

(e) binding the produced TP to a hairpin probe that does not participate in the reaction and synthesizing DNA from the TP by a DNA polymerase to form a dsDNA product;

(f) continuously forming TP from the dsDNA product of step (e) by a cleavage enzyme and a DNA polymerase;

(g) binding the TP formed in step (f) to a hairpin probe that does not participate in the reaction and repeating steps (e) to (g) to amplify the production of a dsDNA product; and

(h) identifying the dsDNA to detect the target nucleic acid.

In the present invention, the DNA polymerase may have strand displacement activity to displace DNA bound to a template.

In the present invention, the detection may be performed by detecting a fluorescence signal generated by binding IP 1 to a fluorescent substance specifically binding to dsDNA to.

In another aspect, the present invention provides a composition for detecting a target nucleic acid comprising a target nucleic acid-containing sample, the hairpin probe, a primer capable of binding to the stem of the hairpin probe, a DNA polymerase, and dNTP.

In the present invention, the DNA polymerase can be used without limitation, as long as it is a DNA polymerase having strand displacement activity, and the DNA polymerase is preferably a Klenow Fragment, Vent (exo-) DNA polymerase, Bst DNA polymerase, phi29 DNA polymerase or the like.

In the present invention, a restriction enzyme (nicking endonuclease) may be used as the DNA cleavage enzyme, and any enzyme that recognizes and cleaves a certain recognition site of the DNA sequence may be used as the DNA cleavage enzyme without limitation.

### Examples

Hereinafter, the present invention will be described in more detail with reference to the following examples. However, it will be obvious to those skilled in the art that the following examples are provided only for illustration of the present invention and should not be construed as limiting the scope of the present invention based on the subject matter of the present invention.

### Example 1: Establishment of Reaction Conditions of Target Nucleic Acid Detection Using Hairpin Probe-Assisted Isothermal Probe Amplification

The process of preparing the HIPAmp reaction solution of the present invention is as follows, but is not limited thereto. The reaction solution (final 50 μL) used in this example was prepared by adding 2 μL of dNTPs (2.5 mM each), 1 μL of a hairpin probe (5 μM), 1 μL of a primer (500 nM), 1 μL of SYBR Green I (50×), and 1 μL of a target nucleic acid to a reaction buffer solution. The reaction buffer solution contained 10 mM Tris-HCl (pH 7.9), 50 mM NaCl, 10 mM MgCl₂, 0.1 mM DTT, 11.4%, and 90 μg/mL of BSA. The reaction solution thus prepared was preheated at 37° C. for 20 minutes, 1 μL of Klenow fragment (3′-5′ exo-) (2 unit/μL) and 0.5 μL of Nt.AlwI (6 unit/μL) were added to the reaction solution, and the reaction was allowed to proceed at 37° C. for 2 hours. During the reaction, the fluorescence signal generated from SYBR Green I bound to the dsDNA product was measured at 30-second intervals to analyze the amount of the dsDNA product in real time.

In this example, the base sequence information of the DNA that was used is as follows, but is not limited thereto.

Hairpin probe:
(SEQ ID NO: 1)
5′-CTG TTC TGC CCC TTC TTT TGA T [symbol] GGT TGA GCT
GCT GAG AGT CCA AGA GTG CTC AAC CGG CTG TTC TGC
CCC TTC-3′

In the base sequence of the hairpin probe, the base sequences indicated by underlining, italics, and boldface represent the base sequences of the cCP, the cleavage enzyme recognition site and the hairpin stem, respectively.

Primer:
(SEQ ID NO: 2)
5′-CCG GTT GAG C-3′
Target nucleic acid:
(SEQ ID NO: 3)
5′-GGT CTT ACA CAA GAG GAC TCT TGG ACT CTC AGC
AAT GTC AAC GAC C-3′

### Example 2. Verification of Novel Concept of Target Nucleic Acid Detection Reaction System Using Hairpin Probe-Assisted Isothermal Probe Amplification

Experiments for verifying the reaction system of HIPAmp were conducted using the reaction conditions mentioned in Example 1.

The experiments were conducted under the following five reaction conditions.

1: Reaction conditions excluding the addition of a hairpin probe and primer;

2: Reaction conditions excluding the addition of a hairpin probe;

3: Reaction conditions excluding the addition of a primer;

4: Reaction conditions including the addition of both a hairpin probe and a primer;

5: Reaction conditions excluding the addition of target nucleic acid.

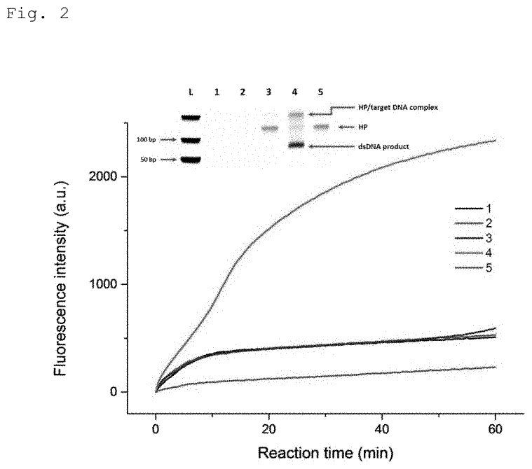

The result of the determination as to whether or not a fluorescence signal was generated depending on the addition of a hairpin probe and a primer showed that a strong fluorescence signal was generated only in a sample in which both the hairpin probe and the primer were added, as shown in FIG. 2.

In addition, it was found that, when the target nucleic acid was not present, no fluorescence signal was generated even though both the hairpin probe and the primer were added. The reaction system verification experiment using electrophoresis showed the same tendency as the result of the reaction system verification experiment based on the fluorescence signal.

### Example 3. Verification of Sensitivity of Novel Concept of Target Nucleic Acid Detection Method Using Hairpin Probe-Assisted Isothermal Probe Amplification

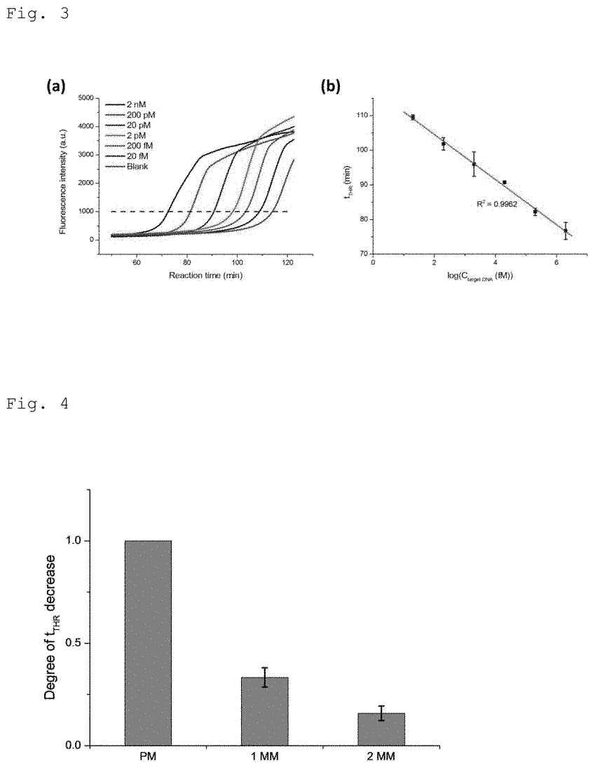

An experiment for verifying the sensitivity of HIPAmp was performed using the reaction conditions used in Example 1. Analytical samples containing target nucleic acids of various concentrations (20 fM to 2 nM) were prepared and then a HIPAmp reaction was conducted. As shown in FIG. 3, the result showed that the limit of detection (LOD) of the target nucleic acid according to the present method was 0.33 fM.

### Example 4. Verification of Selectivity of Novel Concept of Target Nucleic Acid Detection Method Using Hairpin Probe-Assisted Isothermal Probe Amplification

An experiment for verifying the selectivity of HIPAmp was performed using the reaction conditions used in Example 1. In the experiment for verifying selectivity, the degrees of t_{THR} decrease measured for samples containing various types of target nucleic acids were compared, but the present invention is not limited thereto.

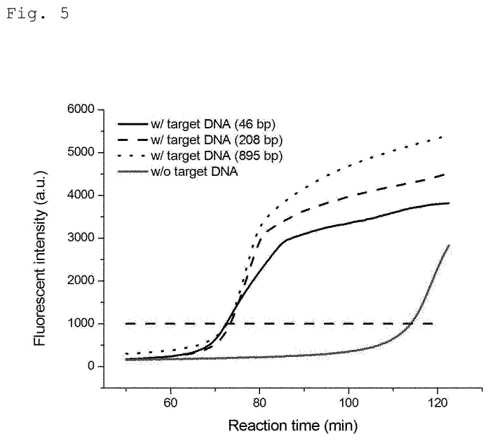

An analytical sample containing a target nucleic acid having a mismatched part was prepared, and then a HIPAmp reaction was performed. As shown in FIG. 4, the result showed that the degree of t_{THR} decrease was significantly reduced. The result of this experiment showed that the proposed HIPAmp was capable of detecting even a single-base mismatch contained in the target nucleic acid.

The degree of t_{THR} decrease may be defined by the following Equation.

Degree of t_{THR} decrease=(t_{THR.NC}−t_{THR.T})/(t_{THR.NC}−t_{THR.PM})

Each variable used in Equation above is defined as follows: t_{THR.NC}: t_{THR} measurement value of a sample containing a non-complementary target nucleic acid; t_{THR.PM}: t_{THR} measurement value of a sample containing a perfectly matched target nucleic acid; and t_{THR.T}: t_{THR} measurement value of a sample containing different types of target nucleic acids (non-complementary, mismatched, and perfectly matched).

### Example 5. Verification of Practicality of Target Nucleic Acid Detection Method Using Hairpin Probe-Assisted Isothermal Probe Amplification

In order to verify the practicality of HIPAmp, t_{THR} values measured from samples containing different lengths of target nucleic acids were compared. Samples containing a target nucleic acid (2 nM) of different lengths (46, 208, and 895 bp) were prepared and then a HIPAmp reaction was performed. As shown in FIG. 5, the result showed that the t_{THR} measurement values of respective samples were almost identical.

The results of this experiment demonstrated that the HIPAmp proposed by the present invention can solve the problem of limitation of the length of the target nucleic acid in conventional EXPAR.

### INDUSTRIAL AVAILABILITY

The present invention has an effect of performing detection on a wide range of target nucleic acids by solving the problem of limited utilization range of target nucleic acids of EXPAR, which is a conventional isothermal probe amplification technique.

Although specific configurations of the present invention have been described in detail, those skilled in the art will appreciate that the preferred embodiments are given for merely illustrative purposes in the description and should not be construed as limiting the scope of the present invention. Therefore, the substantial scope of the present invention is defined by the accompanying claims and equivalents thereto.

### SEQUENCE LISTING FREE TEXT

An electronic file is attached.

1. A hairpin probe for detecting a target nucleic acid comprising:
(i) a hairpin ring having a base sequence specifically capable of binding to a target nucleic acid;
(ii) hairpin stems having a base sequence capable of binding to a primer and being capable of complementarily binding to each other, wherein each hairpin stem is linked to each end of the hairpin ring;
(iii) cTPs linked to each end of hairpin stems of (ii) and having a sequence complementary to a trigger probe (TP); and
(iv) a cleavage enzyme recognition base sequence located between the hairpin stem and the cTP of a 5′ end of the hairpin probe.

2. A method for detecting a target nucleic acid comprising:
(a) reacting a composition comprising the hairpin probe according to claim 1, a primer capable of binding to the stem of the hairpin probe, a DNA polymerase and dNTP with a target nucleic acid-containing sample to amplify dsDNA; and
(b) analyzing the amplified dsDNA to detect a target nucleic acid.

3. The method according to claim 2, wherein the DNA polymerase has strand displacement activity to displace DNA bound to a template.

4. The method according to claim 2, wherein the detection is performed by detecting a fluorescence signal by binding to a fluorescent substance specifically binding to dsDNA.

5. A composition for detecting a target nucleic acid comprising:
the hairpin probe according to claim 1;
a primer capable of binding to the stem of the hairpin probe;
a DNA polymerase; and
dNTP.
